(19) United States
(12) Patent Application Publication
(10) Pub. No.: US 2021/0237086 A1
(43) Pub. Date: Aug. 5, 2021

(54) CHIP FOR GENE SEQUENCING AND GENE SEQUENCING METHOD

(71) Applicants: BEIJING BOE OPTOELECTRONICS TECHNOLOGY CO., LTD., Beijing (CN); BOE TECHNOLOGY GROUP CO., LTD., Beijing (CN)

(72) Inventors: Yue GENG, Beijing (CN); Fengchun PANG, Beijing (CN); Peizhi CAI, Beijing (CN); Le GU, Beijing (CN)

(73) Assignees: BEIJING BOE OPTOELECTRONICS TECHNOLOGY CO., LTD., Beijing (CN); BOE TECHNOLOGY GROUP CO., LTD., Beijing (CN)

(21) Appl. No.: 16/098,674

(22) PCT Filed: Apr. 16, 2018
(86) PCT No.: PCT/CN2018/083225

§ 371 Date: Nov. 2, 2018

(30) Foreign Application Priority Data
May 5, 2017 (CN) 201710313705.2

(51) Int. Cl. B01L 7/00; C12Q 1/6869; G01B 7/16; G01K 7/16

(52) U.S. Cl. CPC B01L 7/52; C12Q 1/6869; G01B 7/18; B01L 2300/0663; B01L 2300/0816; B01L 2300/08; B01L 2300/18; G01K 7/16

(57) ABSTRACT

A chip for gene sequencing and a gene sequencing method are disclosed. The chip for gene sequencing includes: a body, including an accommodating chamber and a temperature testing element, wherein the temperature testing element is configured for testing a temperature variation amount in the accommodating chamber.

### CROSS REFERENCE

The present application claims priority of China Patent application No. 201710313705.2 filed on May 5, 2017, the content of which is incorporated in its entirety as portion of the present application by reference herein.

### TECHNICAL FIELD

Embodiments of the present disclosure relate to a chip for gene sequencing and a gene sequencing method.

### BACKGROUND

Existing gene sequencing is a process of performing different fluorophore modifications (i.e., fluorescence labeling) on various bases in a test reagent, and detecting a fluorescence color released by the fluorophore with an optical system after these bases are paired with a gene fragment to be tested (i.e., a deoxyribonucleotide chain), so that types and the number of the bases of the gene fragment to be tested can be determined, whereby a sequence of the gene fragment to be tested is obtained correspondingly.

### SUMMARY

At least one embodiment of the present embodiment provides a chip for gene sequencing, including: a body, including an accommodating chamber and a temperature testing element, the temperature testing element is configured for testing a temperature variation amount in the accommodating chamber.

For example, in the chip for gene sequencing provided by an embodiment of the present disclosure, the temperature testing element is provided in the accommodating chamber, for testing the temperature variation amount in the accommodating chamber.

For example, in the chip for gene sequencing provided by an embodiment of the present disclosure, the temperature testing element includes: a cantilever beam, on a cavity wall of the accommodating chamber and configured to be thermally deformable; and a deformation detecting element, on the cantilever beam and configured to detect a deformation amount of the cantilever beam to reflect the temperature variation amount of the accommodating chamber, an orthogonal projection of the cantilever beam on a plane where a bottom surface of the accommodating chamber is located is at least partially overlapped with the bottom surface of the accommodating chamber.

For example, in the chip for gene sequencing provided by an embodiment of the present disclosure, the cantilever beam includes: a first base beam and a second base beam arranged side by side; and a connection layer between the first base beam and the second base beam, thermal expansion coefficients of the first base beam and the second base beam are different.

For example, in the chip for gene sequencing provided by an embodiment of the present disclosure, the deformation detecting element includes a piezoresistor, and the piezoresistor is configured to detect the deformation amount of the cantilever beam and reflect the temperature variation amount of the accommodating chamber as a variation amount of a resistance value.

For example, in the chip for gene sequencing provided by an embodiment of the present disclosure, the cantilever beam includes: a first base beam and a second base beam arranged side by side, and a dielectric layer between the first base beam and the second base beam, the first base beam and the second base beam are connected together through the dielectric layer, the deformation detecting element includes a capacitor constituted by the dielectric layer, the first base beam, and the second base beam, and the capacitor is configured to detect the deformation amount of the cantilever beam and reflect the temperature variation amount of the accommodating chamber as a variation amount of a capacitance value.

For example, in the chip for gene sequencing provided by an embodiment of the present disclosure, thermal expansion coefficients of the first base beam and the second base beam are different.

For example, in the chip for gene sequencing provided by an embodiment of the present disclosure, the deformation detecting element further includes a first capacitance test electrode connected with the first base beam and a second capacitance test electrode connected with the second base beam.

For example, in the chip for gene sequencing provided by an embodiment of the present disclosure, the deformation detecting element is at one end of the cantilever beam away from a cavity wall of the accommodating chamber.

For example, in the chip for gene sequencing provided by an embodiment of the present disclosure, one of the first base beam and the second base beam is made of metal aluminum and the other of the first base beam and the second base beam is made of non-metallic silicon.

For example, in the chip for gene sequencing provided by an embodiment of the present disclosure, the cantilever beam is transversely provided at a top end of the accommodating chamber.

For example, in the chip for gene sequencing provided by an embodiment of the present disclosure, the first base beam and the second base beam are arranged side by side in a direction perpendicular to the bottom surface of the accommodating chamber, and an orthogonal projection of the first base beam on the plane where the bottom surface of the accommodating chamber is located is at least partially overlapped with an orthogonal projection of the second base beam on the plane where the bottom surface of the accommodating chamber is located.

For example, in the chip for gene sequencing provided by an embodiment of the present disclosure, the body includes a plurality of accommodating chambers and a plurality of temperature testing elements, the plurality of accommodating chambers are provided in one-to-one correspondence with the plurality of temperature testing elements.

For example, in the chip for gene sequencing provided by an embodiment of the present disclosure, the body further includes: an inlet; an outlet; and an overflow chamber, in communication with the plurality of accommodating chambers; the inlet and the outlet are both in communication with the overflow chamber.

For example, in the chip for gene sequencing provided by an embodiment of the present disclosure, the body includes: a first substrate; a second substrate, the first substrate and the second substrate being provided opposite to each other; and an annular wall, between the first substrate and the second substrate, one end of the annular wall being connected with an edge of the first substrate, and the other end of the annular wall being connected with an edge of the second substrate, one of the first substrate and the second substrate is provided with the plurality of accommodating chambers thereon, the other one of the first substrate and the second substrate is provided with the inlet and the outlet thereon, the overflow chamber is formed on an inner side of the annular wall, and sides of the plurality of accommodating chambers facing the overflow chamber are in communication with the overflow chamber.

For example, in the chip for gene sequencing provided by an embodiment of the present disclosure, a circumscribed circle of a cross section of the accommodating chamber has a diameter of D, and the accommodating chamber has a height of 1.25 D to 5 D.

For example, in the chip for gene sequencing provided by an embodiment of the present disclosure, the diameter D of the circumscribed circle of the cross section of the accommodating chamber is 10 μm to 100 μm.

At least one embodiment of the present disclosure provides a gene sequencing method, including: placing a sample to be tested into an accommodating chamber; adding a test reagent for a base pairing reaction to the accommodating chamber; and testing a temperature variation amount of the accommodating chamber for gene sequencing.

For example, in the gene sequencing method provided by an embodiment of the present disclosure, testing the temperature variation amount of the accommodating chamber for gene sequencing includes: determining whether or not the base pairing reaction occurs and a number of pairing reactions according to a magnitude of the temperature variation amount; and determining a type of a base on the sample to be tested according to a type of a base of the test reagent.

### BRIEF DESCRIPTION OF THE DRAWINGS

In order to clearly illustrate the technical solution of embodiments of the present disclosure, the drawings of the embodiments or related technical description will be briefly described in the following; it is obvious that the drawings in the description are only related to some embodiments of the present disclosure and not limited to the present disclosure.

FIG. 1 is a schematic diagram of a stereoscopic structure of a chip for gene sequencing provided by an embodiment of the present disclosure;

FIG. 2 is a schematic cross-sectional diagram of a main view structure of the chip for gene sequencing shown in FIG. 1;

FIG. 3 is a schematic cross-sectional diagram of a main view structure showing a magnetic bead located in an accommodating chamber provided by an embodiment of the present disclosure;

FIG. 4 is a schematic cross-sectional diagram of a top view structure showing a magnetic bead located in the accommodating chamber provided by an embodiment of the present disclosure;

FIG. 5 is a schematic structural diagram of the cantilever beam shown in FIG. 2; and

FIG. 6 is another schematic structural diagram of the cantilever beam shown in FIG. 2.

### DETAILED DESCRIPTION

In order to make objects, technical details and advantages of the embodiments of the present disclosure apparently, the technical solutions of the embodiments will be described in a clearly and fully understandable way in connection with the drawings related to the embodiments of the present disclosure. It is obvious that the described embodiments are just a part but not all of the embodiments of the present disclosure. Based on the described embodiments herein, a person having ordinary skill in the art may obtain other embodiment(s), without any inventive work, which should be within the scope of the disclosure.

Unless otherwise defined, the technical terms or scientific terms used herein have the same meanings as commonly understood by one of ordinary skill in the art to which the present disclosure belongs. The terms “first,” “second,” etc., which are used in the description and the claims of the present disclosure, are not intended to indicate any sequence, amount or importance, but distinguish various components. The terms “comprises,” “comprising,” “include,” “including,” etc., are intended to specify that the elements or the objects stated before these terms encompass the elements or the objects and equivalents thereof listed after these terms, but do not preclude the other elements or objects. “On,” “under,” “right,” “left” and the like are only used to indicate relative position relationship, and when the position of the object which is described is changed, the relative position relationship may be changed accordingly.

At present, although a testing method of fluorophore modification is a mainstream method in a field of gene sequencing. However, in the testing method, fluorescence labeling with different colors is performed on the four types of bases, while a gene sequencing process generally requires over a thousand of rounds of base pairings, resulting in a larger amount of bases that require fluorophore modifications, which leads to relatively high costs of sequencing reagents, and is not favorable for popularizing and promoting gene sequencing in medicine and other fields.

Embodiments of the present disclosure provide a chip for gene sequencing and a gene sequencing method. The chip for gene sequencing includes: a body, including an accommodating chamber and a temperature testing element, the temperature testing element is configured for testing a temperature variation amount in the accommodating chamber. Therefore, the chip for gene sequencing may perform gene sequencing by directly testing heat generated by base pairing reaction, without performing fluorescence labeling with different colors on the four types of bases, so as to reduce costs of sequencing, and is favorable for popularizing and promoting gene sequencing.

Hereinafter, the chip for gene sequencing and the gene sequencing method provided by the embodiments of the present disclosure will be described with reference to the accompanying drawings.

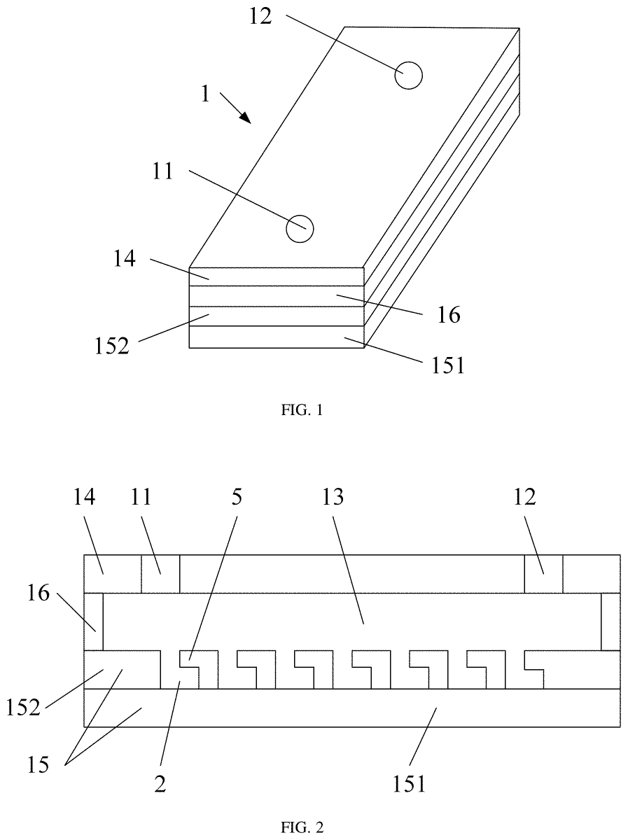

An embodiment of the present disclosure provides a chip for gene sequencing. As illustrated by FIG. 1 and FIG. 2, the chip for gene sequencing includes a body 1, the body 1 including an accommodating chamber 2 and a temperature testing element 3, the temperature testing element 3 is configured for testing a temperature variation amount in the accommodating chamber 2.

In a case where the chip for gene sequencing provided by the embodiment of the present disclosure is configured for performing gene sequencing, in the sequencing procedure, heat released by complementary pairing of bases producing a phosphoester bond and a hydrogen bond results in rise of a temperature of a reaction environment (i.e., a temperature of the accommodating chamber 2), the temperature testing element 3 determines a type and a number of the paired bases by testing the temperature variation amount in the accommodating chamber 2, and sequentially tests the types and numbers of subsequent paired bases by using the same method, so as to obtain a sequence of the gene to be tested. Heat generated by pairing may be reflected through the temperature variation amount in the accommodating chamber 2 as tested by the temperature testing element 3.

Therefore, the chip for gene sequencing provided by the embodiment of the present disclosure may perform gene sequencing by directly testing heat generated by base pairing reaction, without performing fluorescence labeling with different colors on the four types of bases, so as to reduce costs of sequencing, and is favorable for popularizing and promoting gene sequencing. In addition, the chip for gene sequencing has a simple structure, with a convenient sequencing operation.

For example, a circumscribed circle of a cross section of the accommodating chamber 2 has a diameter D of 10 μm to 100 μm, the accommodating chamber 2 has a height of 1.25 D to 5 D, and the cross section of the accommodating chamber 2 may be a circle or a regular hexagon, and the like.

For example, a magnetic bead 4 for carrying a sample to be tested (for example, a deoxyribonucleotide chain) may be provided in the accommodating chamber 2. A size of the accommodating chamber 2 is adapted to that of the magnetic beads 4, to ensure that one accommodating chamber 2 may accommodate only one magnetic bead 4.

For example, one magnetic bead 4 has only one kind of deoxyribonucleotide chain thereon (but this kind of deoxyribonucleotide chain may be replicated into a plurality of deoxyribonucleotide chains on a surface of the magnetic bead).

For example, in some examples, the temperature testing element 3 is provided within the accommodating chamber 2, so that a temperature variation in the accommodating chamber 2 can be tested. Moreover, this configuration can prevent the base pairing reactions in other accommodating chamber from affecting the temperature testing element 3.

For example, as illustrated by FIG. 2 to FIG. 4, the body 1 further has an inlet 11 and an outlet 12 thereon; the interior of the body 1 has an overflow chamber 13, a plurality of accommodating chambers 2 and a plurality of temperature testing elements 3; the plurality of accommodating chamber 2, the inlet 11 and the outlet 12 are all connected with the overflow chamber 13; and the plurality of temperature testing elements 3 are provided in the plurality of accommodating chambers 2 in one-to-one correspondence, for testing temperature variations in the plurality of accommodating chambers 2 in one-to-one correspondence. In this way, gene sequencings of a plurality of deoxyribonucleotide chains can be simultaneously implemented, and the sequencing efficiency can be improved.

For example, as illustrated by FIG. 2 to FIG. 4, the magnetic beads 4 may enter the overflow chamber 13 through the inlet 11, and then enter the accommodating chamber 2 from the overflow chamber 13; because one accommodating chamber 2 may only accommodate one magnetic bead 4, the plurality of magnetic beads 4 may enter the plurality of accommodating chambers 2 in one-to-one correspondence, the accommodating chambers 2 are independent from each other, and heat released from the reaction in the accommodating chamber will not be transferred to one another. During sequencing, firstly, one type of test reagent is applied from the inlet 11 into the overflow chamber 13, and then flows into the accommodating chambers 2, temperature variation in each of the accommodating chambers 2 is tested by its respective temperature testing element 3; if bases of deoxyribonucleotide chains in an accommodating chamber 2 are not paired, then a temperature in the accommodating chamber 2 does not vary; if bases of deoxyribonucleotide chains in an accommodating chamber 2 are paired, then a temperature in the accommodating chamber 2 varies; the number of bases may be obtained according to the temperature variation amount, and the type of the bases may be obtained according to the type of the applied test reagent, which are correspondingly recorded one by one; after the test is completed, the test reagent in the chip is cleaned, and replaced by another test reagent, and types of bases are tested and recorded again, until deoxyribonucleotide chains in all the accommodating chambers 2 are sequenced.

For example, in all sequencing processes, a minimum temperature variation amount generated by base pairing within a single accommodating chamber 2 should be an amount of heat released upon a single base being paired (a single base on the deoxyribonucleotide chain in the accommodating chamber 2), whereby, upon there being a plurality of consecutive identical bases on the deoxyribonucleotide chain, the number of the plurality of identical bases may be derived (which may be calculated according to an amount of heat released upon the plurality of consecutive identical bases are subjected to pairing reactions).

For example, in some examples, as illustrated by FIG. 2 to FIG. 4, the body 1 includes a plurality of accommodating chambers 2 and a plurality of temperature testing elements 3, and the plurality of accommodating chambers 2 are provided in one-to-one correspondence with the plurality of temperature testing elements 3, so as to implement high throughput sequencing.

For example, in some examples, as illustrated by FIG. 1 and FIG. 2, the body 1 includes: a first substrate 14 (which may be made of glass, silicon, polymer, etc.), a second substrate 15 (which may be made of silicon, etc.), the first substrate 14 and the second substrate 15 are provided opposite to each other; and an annular wall 16 (which may be made of silicon oxide, silicon nitride, polymer, etc.), between the first substrate 14 and the second substrate 15, one end of the annular wall is connected with an edge of the first substrate 14, and the other end of the annular wall is connected with an edge of the second substrate 15; the second substrate 15 is provided thereon with the plurality of accommodating chambers 2, the first substrate 14 is provided thereon with the inlet 11 and the outlet 12, the overflow chamber 13 is formed on an inner side of the annular walls 16, and sides of the plurality of accommodating chambers 2 facing the overflow chamber 13 are in communication with the overflow chamber 13.

Of course, as illustrated by FIG. 1 to FIG. 3, the second substrate 15 may also be of a composite structure, which, for example, includes a glass substrate layer 151 and a silicon etching layer 152; the silicon etching layer 152 is provided on the glass substrate layer 151, the accommodating chamber 2 is etched on the silicon etching layer 152, and the cantilever beam 5 is transversely provided at an upper opening of the accommodating chamber 2.

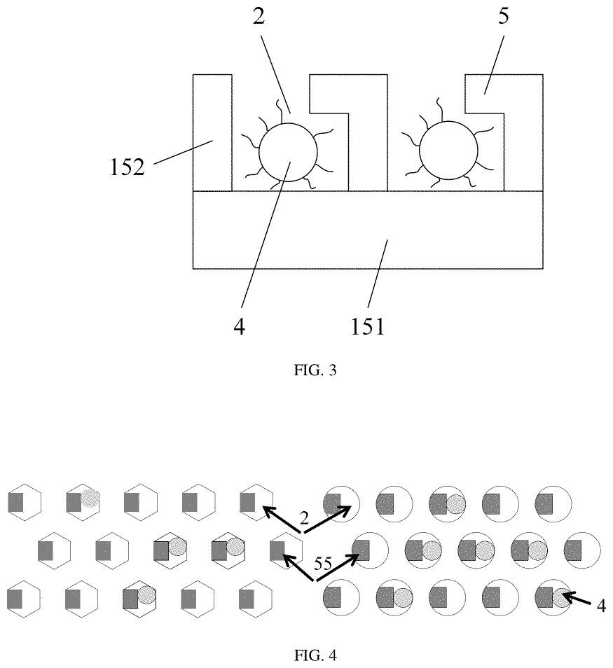

For example, in some examples, as illustrated by FIG. 2 to FIG. 6, the temperature testing element 3 includes: a cantilever beam 5, provided on a cavity wall of the accommodating chamber 2 and configured to be thermally deformable; and a deformation detecting element 30 on the cantilever beam 5, configured to detect a deformation amount of the cantilever beam 5, to reflect the temperature variation amount of the accommodating chamber 2, and an orthogonal projection of the cantilever beam 5 on a plane where a bottom surface of the accommodating chamber 2 is located is at least partially overlapped with a bottom surface of the accommodating chamber 2. Thus, the cantilever beam 5 may convert the temperature variation into deformation, and the deformation detecting element 30 may reflect the temperature variation amount of the accommodating chamber 2, so as to achieving testing a tiny temperature variation amount, and then some parameters (resistance, capacitance) of the deformation detecting element 30 are detected, so that the temperature variation amount generated in the corresponding accommodating chamber 2 can be obtained. It should be noted that, reference number 55 denotes an upright post of the cantilever beam 5, which is not shown by cross section lines in FIG. 2 and FIG. 3.

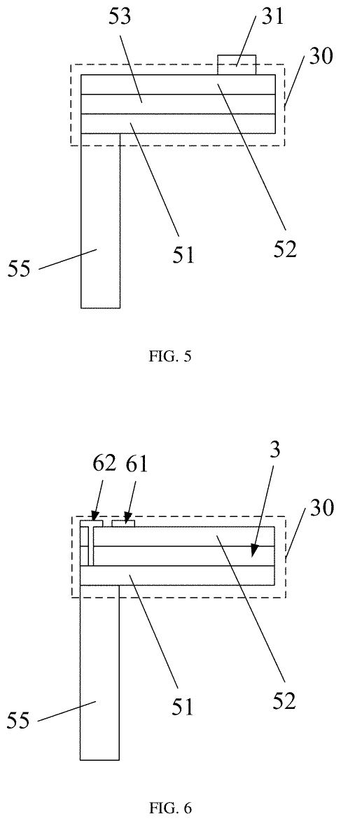

For example, in some examples, as illustrated by FIG. 5, the cantilever beam 5 includes: a first base beam 51 and a second base beam 52 arranged side by side; and a connection layer 53 provided between the first base beam 51 and the second base beam 52, thermal expansion coefficients of the first base beam 51 and the second base beam 52 are different. Therefore, upon the temperature varying, the first base beam 51 and the second base beam 52 are heated to cause a thermal expansion coefficient mismatch, so that thermal stress is generated to cause the first base beam 51 and the second base beam 52 to be bent and deformed, and the connection layer 53 is configured for preferably connecting the first base beam 51 with the second base beam 52, to prevent the first base beam 51 and the second base beam 52 from peeling off due to the thermal expansion coefficient mismatch.

For example, in some examples, the deformation detecting element 30 includes a piezoresistor 31, and the piezoresistor 31 is configured to detect the deformation amount of the cantilever beam 5 and reflect the temperature variation amount of the accommodating chamber 2 as a variation amount of a resistance value. Thus, the deformation amount of the cantilever beam 5 may be converted by the piezoresistor 31 into the variation amount of the resistance value detectable by an electric signal; and because the resistance value may be accurately measured, it is easy to achieving testing a tiny temperature variation amount; in addition, during the testing process, it may also be directly converted into a data signal, for a processor to process automatically.

For example, in some examples, as illustrated by FIG. 5, the deformation detecting element 30, for example, the piezoresistor 31, is provided at one end of the cantilever beam 5 away from the cavity wall of the accommodating chamber 2. Thus, because the deformation amount generated by the end of the cantilever beam 5 away from the accommodating chamber 2 is relatively large, the deformation detecting element 30 may detect the deformation amount of the cantilever beam 5 more easily.

For example, in some examples, as illustrated by FIG. 6, the cantilever beam 5 includes: the first base beam 51 and the second base beam 52 arranged side by side, and a dielectric layer 54 provided between the first base beam 51 and the second base beam 52; the first base beam 51 and the second base beam 52 are connected together through the dielectric layer 54, the deformation detecting element 30 includes a capacitor constituted by the dielectric layer 54, the first base beam 51 and the second base beam 52, and the capacitor is configured to detect the deformation amount of the cantilever beam 5 and reflect the temperature variation amount of the accommodating chamber 2 as a variation amount of a capacitance value. Upon the cantilever beam 5 being heated, a dielectric constant of the dielectric layer 54 varies, and a thickness of the dielectric layer 54 varies, causing a separation distance between the first base beam 51 and the second base beam 52 to vary, which results in a variation of the capacitance value; and the temperature variation amount is determined according to the variation amount of the capacitance value. Thus, the deformation amount of the cantilever beam 5 may be converted by the capacitor constituted by the dielectric layer 54 with the first base beam 51 and the second base beam 52 into the variation amount of the capacitance value detectable by an electric signal; and because the capacitance value may be accurately measured, it is easy to achieving testing a tiny temperature variation amount; in addition, during the testing process, it may also be directly converted into a data signal, for the processor to process automatically.

For example, the thermal expansion coefficients of the first base beam 51 and the second base beam 52 may be the same or different; upon the thermal expansion coefficients of the first base beam 51 and the second base beam 52 being different from each other, the cantilever beam 5 is thermally bent, the dielectric constant of the dielectric layer 54 varies, the thickness of the dielectric layer 54 varies, and curvatures of the first base beam 51 and the second base beam 52 are also different from each other, causing the separation distance between the first base beam 51 and the second base beam 52 to vary, which results in a variation of the capacitance value; and the temperature variation amount is determined according to the variation amount of the capacitance value. For example, one of the first base beam 51 and the second base beam 52 is made of metal aluminum, and the other one of the first base beam 51 and the second base beam 52 is made of non-metal silicon, thermal expansion coefficients of the first base beam 51 and the second base beam 52 are significantly different; and upon a temperature varying, the first base beam 51 and the second base beam 52 are heated to cause a thermal expansion coefficient mismatch, so that thermal stress is generated to cause the cantilever beam 5 to be bent and deformed.

For example, in some examples, as illustrated by FIG. 6, the deformation detecting element 30 further includes a first capacitance test electrode 61 connected with the first base beam 51 and a second capacitance test electrode 62 connected with the second base beam 52. Therefore, the capacitance value of the capacitor constituted by the dielectric layer 54, the first base beam 51 and the second base beam 52 may be detected by the first capacitance test electrode 61 and the second capacitance test electrode 62.

For example, in some examples, as illustrated by FIG. 5 and FIG. 6, the cantilever beam 5 is transversely provided at a top end of the accommodating chamber 2. Therefore, the cantilever beam 5 can hinder dissipation of heat in the accommodating chamber 2, and is more favorable for improving test accuracy of the temperature variation in the accommodating chamber 2.

For example, in some examples, as illustrated by FIG. 5 and FIG. 6, the first base beam 51 and the second base beam 52 are arranged side by side in a direction perpendicular to the bottom surface of the accommodating chamber 2, and an orthogonal projection of the first base beam 51 on the plane where the bottom surface of the accommodating chamber 2 is located is at least partially overlapped with an orthogonal projection of the second base beam 52 on the plane where the bottom surface of the accommodating chamber 2 is located.

For example, the orthogonal projection of the first base beam 51 on the plane where the bottom surface of the accommodating chamber 2 is located may be completely overlapped with the orthogonal projection of the second base beam 52 on the plane where the bottom surface of the accommodating chamber 2 is located.

At least one embodiment of the present disclosure further provides a gene sequencing method, including: placing a sample to be tested into an accommodating chamber; adding a test reagent for a base pairing reaction to the accommodating chamber; and testing a temperature variation amount of the accommodating chamber for gene sequencing.

For example, in some examples, the testing a temperature variation amount of the accommodating chamber for gene sequencing includes: determining whether or not the base pairing reaction occurs and a number of pairing reactions according to a magnitude of the temperature variation amount; and determining a type of a base on the sample to be tested according to a type of a base of the test reagent. In the gene sequencing method provided by the present disclosure, the chip for gene sequencing according to any one of the above-described embodiments is configured for sequencing the bases of the deoxyribonucleotide chain, which is specifically provided as follows: bases of the deoxyribonucleotide chain are sequentially tested with four types of test reagents, after the base to be tested of the deoxyribonucleotide chain is paired with the corresponding base of the test reagent, the number of bases to be tested is determined by testing the generated heat, and the type of the base to be tested of the deoxyribonucleotide chain is determined according to the type of the corresponding base of the test reagent, until bases of the entire deoxyribonucleotide chain are tested; there is one type of a single base in any one of the test reagents, and there are four types of single bases in the four types of test reagents.

According to a principle that the bases of the deoxyribonucleotide chain may only be paired one by one, the bases of the deoxyribonucleotide chain are sequentially tested with the four test reagents, and after the base to be tested of the deoxyribonucleotide chain is paired with the corresponding base of the test reagent, the number of bases to be tested is determined by testing the generated heat, the type of the base to be tested of the deoxyribonucleotide chain is determined by the type of the corresponding base of the test reagent, the types of the bases and the number of bases of the reaction may be obtained, then the test reagent for performing the reaction is cleaned, and the types of subsequent bases and the number of paired bases are sequentially tested again by using the same method, until bases of the entire deoxyribonucleotide chain are all tested; there is one type of a single base in any one of the test reagents, and there are four types of single bases in the four types of test reagents.

In a process of replacing a test reagent for testing, it is needed to firstly clean a test reagent selected last time in a pairing environment of the deoxyribonucleotide chain, and then add a test reagent selected this time to the pairing environment of the deoxyribonucleotide chain, in order to ensure test accuracy, and avoid a case where the test reagent selected last time happens to be able to be paired with a gene behind a paired gene after pairing this time.

In summary, the chip for gene sequencing provided by the present disclosure has a simple structure, with a convenient sequencing operation; it is not needed to perform fluorescence labeling on the base upon the gene being sequenced; heat released by complementary pairing of the bases forming a phosphoester bond and a hydrogen bond in the sequencing procedure results in rise of a temperature in the reaction environment, the temperature testing element determines the types of the paired bases and the number of paired bases by testing the temperature variation amount in the accommodating chamber, and sequentially tests the types of subsequent bases and the number of paired by using the same method, so as to obtain the sequence of the gene to be tested.

In the gene sequencing method provided by the present disclosure, according to the principle that the bases of the deoxyribonucleotide chain may only be paired one by one, the bases of the deoxyribonucleotide chains are sequentially tested with the four types of test reagents, and after the base to be tested of the deoxyribonucleotide chain is paired with the corresponding base of the test reagent, the number of bases to be tested is determined by testing generated heat, the type of the base to be tested of the deoxyribonucleotide chain is determined by the type of the corresponding base of the test reagent, the types of the bases and the number of bases of the reaction may be obtained, and the types of subsequent bases and the number of bases paired are sequentially tested again by using the same method, until bases of the entire deoxyribonucleotide chain are all tested; there is one type of a single base in any one of the test reagents, and there are four types of single bases in the four types of test reagents; in a process of replacing a test reagent, it is needed to clean a test reagent selected last time in the pairing environment of the deoxyribonucleotide chain, and then add a test reagent selected this time to the pairing environment of the deoxyribonucleotide chain, in order to ensure test accuracy.

The following points should to be explained:

1) The drawings of at least one embodiment of the present disclosure only relate to the structure in the embodiment of the present disclosure, and other structures may be referenced to the usual design.

2) In the absence of conflict, the features of the same embodiment and the different embodiments ban be combined with each other.

The above are only specific implementations of the present disclosure, however the scope of the present disclosure is not limited thereto, variations or substitutions that easily occur to any one skilled in the art within the technical scope disclosed in the present disclosure should be encompassed in the scope of the present disclosure. Therefore, the scope of the present disclosure should be based on the scope of the claims.

1: A chip for gene sequencing, comprising:
a body, comprising an accommodating chamber and a temperature testing element,
wherein the temperature testing element is configured for testing a temperature variation amount in the accommodating chamber.

2: The chip for gene sequencing according to claim 1, wherein the temperature testing element is provided in the accommodating chamber, for testing the temperature variation amount in the accommodating chamber.

3: The chip for gene sequencing according to claim 1, wherein the temperature testing element comprises:
a cantilever beam, on a cavity wall of the accommodating chamber and configured to be thermally deformable; and
a deformation detecting element, on the cantilever beam and configured to detect a deformation amount of the cantilever beam to reflect the temperature variation amount of the accommodating chamber,
wherein an orthogonal projection of the cantilever beam on a plane where a bottom surface of the accommodating chamber is located is at least partially overlapped with the bottom surface of the accommodating chamber.

4: The chip for gene sequencing according to claim 3, wherein the cantilever beam comprises:
a first base beam and a second base beam arranged side by side; and
a connection layer between the first base beam and the second base beam,
wherein thermal expansion coefficients of the first base beam and the second base beam are different.

5: The chip for gene sequencing according to claim 4, wherein the deformation detecting element comprises a piezoresistor, and the piezoresistor is configured to detect the deformation amount of the cantilever beam and reflect the temperature variation amount of the accommodating chamber as a variation amount of a resistance value.

6: The chip for gene sequencing according to claim 3, wherein the cantilever beam comprises:
a first base beam and a second base beam arranged side by side, and
a dielectric layer between the first base beam and the second base beam,
wherein the first base beam and the second base beam are connected together through the dielectric layer, the deformation detecting element comprises a capacitor constituted by the dielectric layer, the first base beam, and the second base beam, and the capacitor is configured to detect the deformation amount of the cantilever beam and reflect the temperature variation amount of the accommodating chamber as a variation amount of a capacitance value.

7: The chip for gene sequencing according to claim 6, wherein thermal expansion coefficients of the first base beam and the second base beam are different.

8: The chip for gene sequencing according to claim 6, wherein the deformation detecting element further comprises a first capacitance test electrode connected with the first base beam and a second capacitance test electrode connected with the second base beam.

9: The chip for gene sequencing according to claim 3, wherein the deformation detecting element is at one end of the cantilever beam away from a cavity wall of the accommodating chamber.

10: The chip for gene sequencing according to claim 4, wherein one of the first base beam and the second base beam is made of metal aluminum and the other of the first base beam and the second base beam is made of non-metallic silicon.

11: The chip for gene sequencing according to claim 3, wherein the cantilever beam is transversely provided at a top end of the accommodating chamber.

12: The chip for gene sequencing according to claim 4, wherein the first base beam and the second base beam are arranged side by side in a direction perpendicular to the bottom surface of the accommodating chamber, and an orthogonal projection of the first base beam on the plane where the bottom surface of the accommodating chamber is located is at least partially overlapped with an orthogonal projection of the second base beam on the plane where the bottom surface of the accommodating chamber is located.

13: The chip for gene sequencing according to claim 1, wherein the body comprises a plurality of accommodating chambers and a plurality of temperature testing elements, the plurality of accommodating chambers are provided in one-to-one correspondence with the plurality of temperature testing elements.

14: The chip for gene sequencing according to claim 13, wherein, the body further comprises:
an inlet;
an outlet; and
an overflow chamber, in communication with the plurality of accommodating chambers;
wherein the inlet and the outlet are both in communication with the overflow chamber.

15: The chip for gene sequencing according to claim 14, wherein, the body comprises:
a first substrate;
a second substrate, the first substrate and the second substrate being provided opposite to each other; and
an annular wall, between the first substrate and the second substrate, one end of the annular wall being connected with an edge of the first substrate, and the other end of the annular wall being connected with an edge of the second substrate,
wherein one of the first substrate and the second substrate is provided with the plurality of accommodating chambers thereon, the other one of the first substrate and the second substrate is provided with the inlet and the outlet thereon, the overflow chamber is formed on an inner side the annular wall, and sides of the plurality of accommodating chambers facing the overflow chamber are in communication with the overflow chamber.

16: The chip for gene sequencing according to claim 1, wherein a circumscribed circle of a cross section of the accommodating chamber has a diameter of D, and the accommodating chamber has a height of 1.25 D to 5 D.

17: The chip for gene sequencing according to claim 16, wherein the diameter D of the circumscribed circle of the cross section of the accommodating chamber is 10 μm to 100 μm.

18: A gene sequencing method, comprising:
placing a sample to be tested into an accommodating chamber;
adding a test reagent for a base pairing reaction to the accommodating chamber; and
testing a temperature variation amount of the accommodating chamber for gene sequencing.

19: The gene sequencing method according to claim 18, wherein testing the temperature variation amount of the accommodating chamber for gene sequencing comprises:
determining whether or not the base pairing reaction occurs and a number of pairing reactions according to a magnitude of the temperature variation amount; and
determining a type of a base on the sample to be tested according to a type of a base of the test reagent.

20: The chip for gene sequencing according to claim 2, wherein the temperature testing element comprises:
a cantilever beam, on a cavity wall of the accommodating chamber and configured to be thermally deformable; and
a deformation detecting element, on the cantilever beam and configured to detect a deformation amount of the cantilever beam to reflect the temperature variation amount of the accommodating chamber,
wherein an orthogonal projection of the cantilever beam on a plane where a bottom surface of the accommodating chamber is located is at least partially overlapped with the bottom surface of the accommodating chamber.
